(19) United States
(12) Patent Application Publication
(10) Pub. No.: US 2004/0181127 A1
(43) Pub. Date: Sep. 16, 2004

(54) Capsule endoscope system

(75) Inventors: Kazuya Matsumoto, Kamiina-gun (JP); Hiroshi Suzushima, Kamiina-gun (JP); Noriyuki Fujimori, Suwa-shi (JP)

Correspondence Address: SCULLY SCOTT MURPHY & PRESSER, PC, 400 GARDEN CITY PLAZA, GARDEN CITY, NY, 11530

(73) Assignee: Olympus Corporation, Tokyo (JP)

(21) Appl. No.: 10/811,041

(22) Filed: Mar. 26, 2004

(30) Foreign Application Priority Data
Jan. 4, 2003 (JP) 2003-098219

(51) Int. Cl. A61B 1/00

(52) U.S. Cl. 600/101; 600/160

(57) ABSTRACT

A capsule endoscope system includes: a capsule endoscope, of which movement is controlled by a magnetic field externally applied; at least one magnetic-field generation unit for generating a magnetic field focused on one point to control the movement of the capsule endoscope travelling in a body cavity of a subject lying down on an examination table; and a table drive unit for moving the examination table relative to the magnetic-field generation unit.

This application claims benefit of Japanese Application No. 2003-98219 filed on Apr. 1, 2003, the contents of which are incorporated by this reference.

### BACKGROUND OF THE INVENTION

1. Field of the Invention

The present invention relates to a capsule endoscope system having a capsule endoscope shaped like a tablet, the capsule endoscope incorporating an observation unit.

2. Description of the Related Art

In examinations of, for example, body cavities, endoscope systems are generally in practical use and are widespread. Each endoscope system includes various devices such as an endoscope, a light source, an image processor, and a display device.

The endoscope includes, for example, an insertion tube having an image pickup device at the distal end thereof and an operation unit connected to the insertion tube. A universal cord extending from the insertion tube is connected to the light source and the image processor. The insertion tube is inserted into, for example, the mouth of a subject and is advanced through a body cavity thereof to observe a desired region.

In the above-mentioned conventional endoscope system, the length of the insertion tube to be inserted into the body cavity is limited. Thus, the range of observation and examination is also limited.

For example, Japanese Unexamined Patent Application Publication No. 7-289504 discloses a capsule endoscope system and includes various proposals.

According to Japanese Unexamined Patent Application Publication No. 7-289504, the capsule endoscope system primarily includes a small endoscope, namely, a capsule endoscope, and a receiving and recording device. The capsule endoscope is shaped like, for example, a tablet and incorporates image pickup means including a photographing optical system, lighting means, communication means, and power supply means. The receiving and recording device has communication means for communicating with the capsule endoscope by radio, and recording means for recording a received signal.

Referring to FIG. 1, according to Japanese Unexamined Patent Application Publication No. 7-289504, a capsule endoscope system 101 primarily includes a capsule endoscope 102, an examination table 103, a table drive unit 104, a receiving unit 105, an image processing unit 106, a gravitational-direction detection unit 107, an arithmetic unit 108, and a table drive control unit 109. The examination table 103 is supported by a pedestal 110.

The capsule endoscope 102 has image pickup means, lighting means, communication means, and a power supply therein. A subject 100, who has swallowed the capsule endoscope 102, lies on, for example, his/her back on the examination table 103. The table drive unit 104 is interposed between the pedestal 110 and the examination table 103, so that the examination table 103 can be tilted toward any direction, for example, forward or backward and to the right or the left. The receiving unit 105 is integrated with the examination table 103 and receives signals outputted from the capsule endoscope 102. The image processing unit 106 receives image signals outputted from the receiving unit 105 and performs predetermined signal processing to the image signals. The gravitational-direction detection unit 107 receives a signal, outputted from a gravity sensor (not shown) included in the capsule endoscope 102, through the receiving unit 105 and then detects the direction of gravity on the basis of the received signal. The arithmetic unit 108 performs a predetermined operation on the basis of signals outputted from the image processing unit 106 and the gravitational-direction detection unit 107, and generates a predetermined signal to set the tilt (also referred to as the attitude) of the examination table 103. The table drive control unit 109 receives the predetermined signal, which is outputted from the arithmetic unit 108 and includes information related to the attitude of the examination table 103, and drives the table drive unit 104 on the basis of the received signal.

According to the conventional capsule endoscope system 101 with the above-mentioned arrangement, the capsule endoscope 102 is swallowed by the subject 100 and is then moved by peristalsis of the gut of the subject 100 and gravity caused by changing the tilt of the examination table 103.

Finally, the capsule endoscope 102 is naturally eliminated from the body of the subject 100 by peristalsis of the gut thereof.

### SUMMARY OF THE INVENTION

Accordingly, it is an object of the present invention to provide a capsule endoscope system including: a capsule endoscope, of which movement is controlled by a magnetic field externally applied; at least one magnetic-field generation unit for generating a magnetic field focused on one point to control the movement of the capsule endoscope travelling in a body cavity of a subject lying down on an examination table; and a moving unit for moving the examination table or the magnetic-field generation unit relative to each other. The magnetic-field generation unit generates a magnetic field focused on one point and the moving unit moves the examination table or the magnetic-field generation unit relative to each other, so that the movement of the capsule endoscope travelling in the body cavity of the subject, lying down on the examination table, can be controlled.

The above and other objects, features and advantages of the invention will become more clearly understood from the following description referring to the accompanying drawings.

### BRIEF DESCRIPTION OF THE DRAWINGS

FIG. 1 is a block diagram of the entire structure of a conventional capsule endoscope system;

FIG. 2 is a block diagram of the entire structure of a capsule endoscope system according to an embodiment of the present invention;

FIG. 3 is a diagram of the arrangement of the present capsule endoscope system viewed from above;

FIG. 4 shows a first example of the arrangement of a magnetic member formed in a portion of a capsule endoscope used in the capsule endoscope system;

FIG. 5 shows a second example of the arrangement of magnetic members formed in portions of the capsule endoscope in the capsule endoscope system;

FIG. 6 is a block diagram of the entire structure of a capsule endoscope system according to a modification of the embodiment;

FIG. 7 is a block diagram of the entire structure of a capsule endoscope system according to another modification of the embodiment;

FIG. 8 shows a first example of the arrangement of magnetic coils arranged in the capsule endoscope of the capsule endoscope system; and

FIG. 9 shows a second example of the arrangement of magnetic coils arranged in the capsule endoscope of the capsule endoscope system.

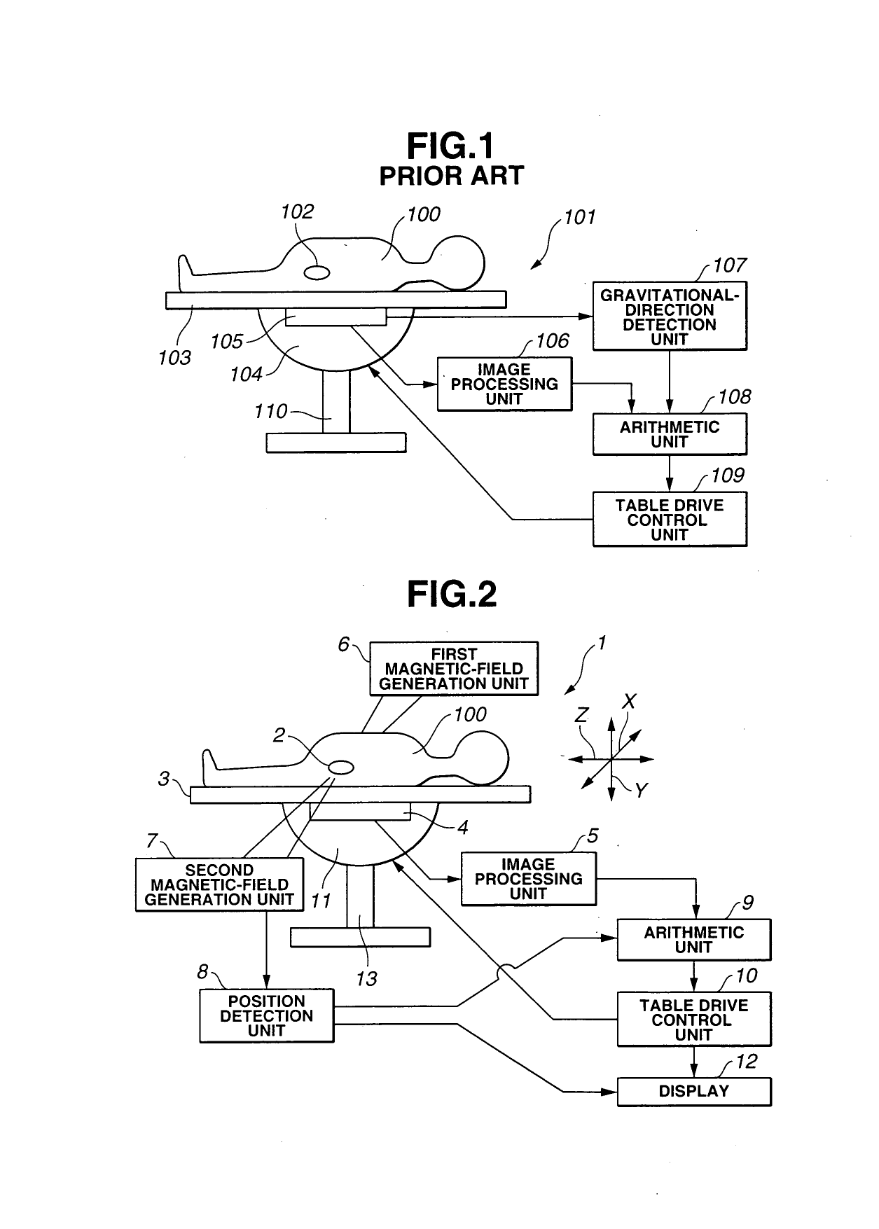

### DESCRIPTION OF THE PREFERRED EMBODIMENT

An embodiment of the present invention will now be described with reference to the drawings.

Referring to FIG. 2, according to the embodiment of the present invention, a capsule endoscope system 1 includes magnetic-field generating means for externally applying magnetic forces to generate a magnetic field focused on one point. A capsule endoscope 2, introduced into a body cavity of a subject, is guided to a desired position in the body cavity of the subject using the magnetic field generated by the magnetic-field generating means, so that a desired portion such as a lesion can be observed at the desired position.

The capsule endoscope system 1 primarily includes the capsule endoscope 2, an examination table 3, a receiving unit 4, an image processing unit 5, a pair of magnetic-field generation units 6 and 7 serving as magnetic-field generating means, a position detection unit 8, an arithmetic unit 9, a table drive control unit 10, a table drive unit 11 serving as moving means, and a display device 12. The examination table 3 is supported by a pedestal 13.

The capsule endoscope 2 includes an image pickup unit, a lighting unit, a communication unit, and a power supply unit, the respective units being not shown. The capsule endoscope 2 is moved and controlled by magnetic fields externally applied.

A subject 100, who has swallowed the capsule endoscope 2, lies on, for example, his/her back on the examination table 3.

The receiving unit 4 is integrated with the examination table 3. The receiving unit 4 receives a predetermined electric signal transmitted from the capsule endoscope 2.

The image processing unit 5 receives the electric signal outputted from the receiving unit 4 and performs predetermined signal processing to the received signal.

The pair of magnetic-field generation units 6 and 7, namely, the first and second magnetic-field generation units 6 and 7 are arranged so as to face each other. The first and second magnetic-field generation units 6 and 7 generate magnetic fields focused on one point.

The position detection unit 8 detects the center of the magnetic fields on the subject 100 on the basis of output signals outputted from the respective magnetic-field generation units 6 and 7.

The arithmetic unit 9 performs a predetermined operation on the basis of a signal outputted from the image processing unit 5 or the position detection unit 8.

The table drive control unit 10 receives a predetermined signal, which is outputted from the arithmetic unit 9 and includes information related to the position of the examination table 3, and then drives the table drive unit 11 on the basis of the received signal. Thus, the position of the subject 100, lying down on the examination table 3, relative to the position of the capsule endoscope 2 is adjusted to control the movement of the capsule endoscope 2 in the body cavity of the subject 100.

The table drive unit 11 is interposed between the pedestal 13 and the examination table 3. Referring to FIG. 2, the table drive unit 11 moves the examination table 3 in the direction shown by the arrow Z, X, or Y relative to the first and second magnetic-field generation units 6 and 7.

The display device 12 displays information related to the position of the capsule endoscope 2 on the basis of the signal outputted from the position detection unit 8, or displays an observed image captured through the capsule endoscope 2 on the basis of the output signals of the image processing unit 5.

A predetermined recording unit for recording captured image signals in a predetermined format is provided for any of the receiving unit 4 and the image processing unit 5 which handle image signals captured by the image pickup unit of the capsule endoscope 2. Alternatively, a predetermined recording device for recording captured image signals in a predetermined format is provided separately from the units 4 and 5. The recording unit or device is not shown in FIG. 2.

The capsule endoscope 2 includes therein various components, namely, the above-mentioned image pickup unit, the lighting unit, the communication unit, and the power supply unit. The structure of the capsule endoscope 2 is not directly related to the present embodiment. Accordingly, the detailed delineation and description of the structure of the capsule endoscope 2 are omitted. As the capsule endoscope 2, a conventional small capsule-shaped endoscope capable of being swallowed into the mouth of the subject 100 can be used.

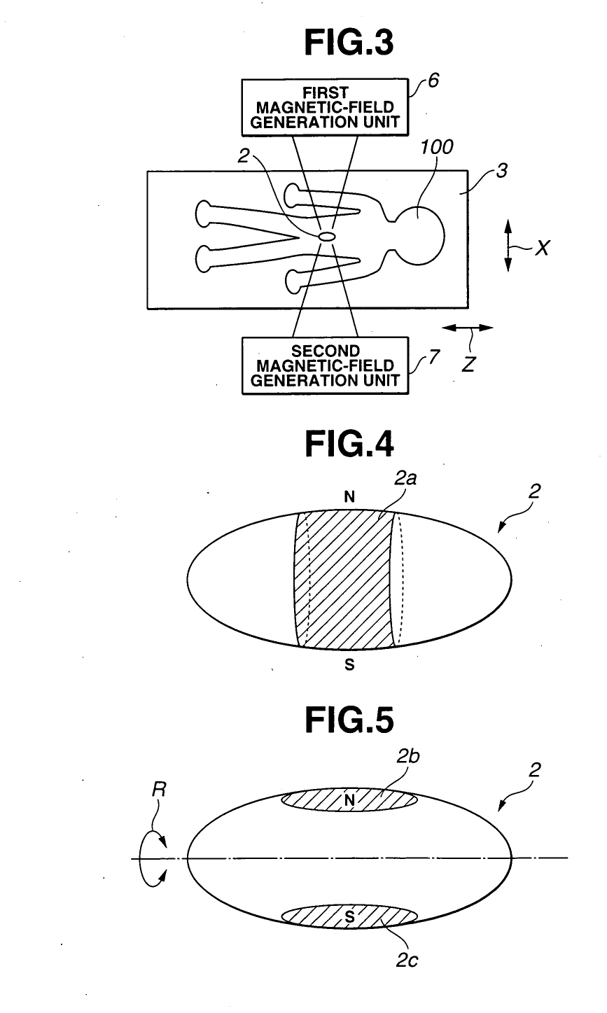

Referring to FIG. 3, in order to generate magnetic fields focused on one point, the first and second magnetic-field generation units 6 and 7 are disposed such that they face each other. The subject 100, who has swallowed the capsule endoscope 2, is arranged between the first and second magnetic-field generation units 6 and 7. Specifically speaking, according to the present embodiment, the first and second magnetic-field generation units 6 and 7 generate magnetic fields from both the sides of the examination table 3 and the subject 100.

According to the present embodiment, the movement of the capsule endoscope 2 is controlled by magnetic forces applied through the magnetic-field generation units 6 and 7. A magnetic member or a magnetic coil serving as a magnetic-field generating member is arranged in at least one portion on the surface of the capsule endoscope 2, or in the interior thereof so that the capsule endoscope 2 is attracted to the applied magnetic fields.

FIG. 4 shows a first example of the arrangement of a magnetic member 2a, serving as the magnetic-field generating member, in a predetermined position on the surface of the capsule endoscope 2. In the first arrangement example, the magnetic member 2a is arranged in the middle on the surface of the capsule endoscope 2. In the first arrangement example, the magnetic member 2a is made of a soft magnetic material such as Permalloy, ferrite, or NiFe. Referring to FIG. 4, the externally-applied magnetic fields have two poles N and S.

FIG. 5 shows a second example of the arrangement of magnetic members 2b and 2c, serving as the magnetic-field generating members, in predetermined positions on the outer or inner surface of the capsule endoscope 2. In the second arrangement example, the magnetic members 2b and 2c are arranged so as to face each other. The magnetic members 2b and 2c are made of a hard magnetic material such as samarium cobalt (Co₅Sm) or neodymium iron boron (Nd₂Fe₁₄B), or a soft magnetic material such as Permalloy, ferrite, or NiFe.

Referring to FIG. 5, the externally-applied magnetic fields have two poles N and S. When a hard magnetic material is used, the magnetic members made of the hard magnetic material can be magnetized previously. In other words, when the hard magnetic material is used, the magnetic members 2b and 2c are previously magnetized so as to have the poles N and S, respectively, as shown in FIG. 5.

Referring to FIGS. 4 and 5, the magnetic member 2a or the magnetic members 2b and 2c are partially arranged in the capsule endoscope 2. Advantageously, the supply of energy consumed for magnetizing the magnetic members arranged in the capsule endoscope 2 is not needed.

Referring to FIGS. 2 and 3, the examination table 3 is movable in the direction Z, X, or Y through the table drive unit 11, which is controlled by the table drive control unit 10.

First, the subject 100, who has swallowed the capsule endoscope 2, is arranged in a range of magnetic fields to be applied through the respective magnetic-field generation units 6 and 7. Subsequently, the magnetic-field generation units 6 and 7 generate magnetic fields focused on one point corresponding to the position of the capsule endoscope 2. The examination table 3 is then moved in a predetermined direction. Consequently, the capsule endoscope 2 can be guided to a desired region such as an affected part or a lesion in the body cavity of the subject 100.

The capsule endoscope 2 can be controlled in various ways using the action of the magnetic fields generated by the magnetic-field generation units 6 and 7. For example, the capsule endoscope 2 can be temporarily held in a desired position in the body cavity. The capsule endoscope 2 can also be temporarily suspended in a predetermined position in an organ such as a stomach having a large internal space.

The display device 12 displays an endoscopic still image or an endoscopic moving image based on image signals, which are captured through the capsule endoscope 2 and are transmitted therefrom. The display device 12 also displays information related to the center of the magnetic fields generated by the magnetic-field generation units 6 and 7, namely, information related to the position of the capsule endoscope 2. The information is based on the output signal of the position detection unit 8.

In the mode of displaying position information, when the magnetic member arranged in the capsule endoscope 2 is made of a hard magnetic material, the position detection unit 8 detects a magnetic field generated by the hard magnetic material to obtain information related to the position of the capsule endoscope 2.

Electric signals photoelectrically converted through the image pickup unit in the capsule endoscope 2 are supplied to the image processing unit 5 through the communication unit (not shown) of the capsule endoscope 2 and the receiving unit 4. The received signals are subjected to predetermined image processing through the image processing unit 5. The resultant image signals are supplied to the display device 12 through the arithmetic unit 9 and the table drive control unit 10. On the basis of the received image signals, an endoscopic still image or an endoscopic moving image is displayed in the display device 12.

In a predetermined case, an electric signal is supplied from the position detection unit 8 to the display device 12. The electric signal outputted from the position detection unit 8 indicates information related to the center of the magnetic fields generated by the magnetic-field generation units 6 and 7, namely, information related to the position of the capsule endoscope 2 in the body cavity of the subject 100. Information based on the electric signal is also displayed in the display device 12.

Therefore, an operator of the capsule endoscope system 1 can obtain information related to the position of the capsule endoscope 2 in the body cavity of the subject 100 and also observe an endoscopic image based on image signals captured by the capsule endoscope 2. In the predetermined case, while observing the endoscopic image and the information, the operator changes the position of the examination table 3 relative to the magnetic-field generation units 6 and 7 by a predetermined operation. Thus, the movement of the capsule endoscope 2 can be controlled.

The operation of the capsule endoscope system 1 with the above-mentioned arrangement will now be described briefly.

First, the subject 100 lies on his/her back on the examination table 3. The examination table 3 is arranged in a predetermined position between the pair of magnetic-field generation units 6 and 7.

Next, the subject 100 swallows the capsule endoscope 2 so that the capsule endoscope 2 travels through the body cavity thereof. Then, the magnetic-field generation units 6 and 7 generate magnetic fields. The subject 100 and the capsule endoscope 2 in the body cavity thereof exist in the magnetic fields generated by the magnetic-field generation units 6 and 7.

The display device 12 displays a predetermined image based on signals of the position detection unit 8 or the image processing unit 5. The operator manually operates, for example, an operating member (not shown) while observing the image displayed in the display device 12, thus allowing the table drive unit 11 to move the examination table 3 in the predetermined direction. Consequently, the capsule endoscope 2 is moved and is then guided to a desired region in the body cavity.

In other words, the capsule endoscope 2 is rapidly moved to a desired region in the body cavity with reliability using magnetic forces as advancing means, the magnetic forces being externally applied to the subject 100.

The movement of the capsule endoscope 2 is controlled manually. In addition, the arithmetic unit 9 performs an arithmetic operation on the basis of the signal of the position detection unit 8 and then supplies the operation result to the table drive control unit 10. The table drive control unit 10 controls the table drive unit 11 on the basis of the operation result to automatically move the examination table 3. Thus, the capsule endoscope 2 can be guided to a desired position.

After the capsule endoscope 2 reaches the desired region, the operator observes the body cavity while adjusting the position of the examination table 3 to control the movement of the capsule endoscope 2. In this instance, the position of the capsule endoscope 2 is flexibly controlled by the magnetic fields generated by the magnetic-field generation units 6 and 7. Therefore, the operator can observe the body cavity while controlling the capsule endoscope 2 so that the capsule endoscope 2 is moved backward, namely, against peristalsis.

After completion of the observation of the desired region in the body cavity, the application of the magnetic forces by the magnetic-field generation units 6 and 7 is interrupted. Accordingly, the capsule endoscope 2 is naturally removed from the body by the peristalsis of the gut.

After completion of the observation, the capsule endoscope 2 can also be guided to, for example, the mouth by controlling the position of the examination table 3. In other words, the capsule endoscope 2 can also be expelled from the body through the mouth.

Instead of swallowing the capsule endoscope 2 as mentioned above, the capsule endoscope 2 can be inserted into the anus of the subject 100. The examination table 3 is moved relative to the magnetic-field generation units 6 and 7, so that the capsule endoscope 2 can be moved from the anus to a desired region.

As mentioned above, according to the present embodiment, the capsule endoscope system 1 is designed such that the magnetic member is arranged in at least one portion of the capsule endoscope 2, the magnetic-field generation units 6 and 7 are arranged, and the examination table 3 is movable. Advantageously, the capsule endoscope 2, existing in the body cavity of the subject 100, can be moved in any direction with a pushing force caused by the action of the magnetic forces without depending on gravity and peristalsis.

Consequently, it is easy to drastically reduce the time required for observation as compared to that for observation with the capsule endoscope 2 which is moved depending on gravity and peristalsis. Thus, binding time of the subject 100 is reduced, resulting in a reduction in burden on the subject 100.

The position of the capsule endoscope 2 in the body cavity can be actively controlled, so that the operator can easily observe a desired region. For example, the operator can perform fixed-point observation while the capsule endoscope 2 is temporarily being held. Even after the capsule endoscope 2 passes a certain region in the body cavity, the operator can easily return the capsule endoscope 2 to the region and again observe it.

Moreover, after completion of the observation of the desired region, the capsule endoscope 2 can be guided by the magnetic forces generated from the magnetic-field generation units 6 and 7, and be rapidly expelled from the body cavity with reliability. On the other hand, the application of the magnetic force generated from the magnetic-field generation units 6 and 7 is interrupted, so that the capsule endoscope 2 can be naturally removed from the body.

Further, the capsule endoscope 2 can be introduced into either the mouth or the anus of a subject. Accordingly, the capsule endoscope 2 can more rapidly reach a region to be observed. After completion of the observation, the capsule endoscope 2 can be expelled from either the mouth or the anus, resulting in the reduction of observation time.

As mentioned above, the total observation time can be reduced. In consideration of the capacity of a power supply of the capsule endoscope 2, therefore, the capacity of the image pickup unit to capture image signals can be easily increased. The image pickup unit is included in the capsule endoscope 2.

Generally, the quality of moving images captured through an image pickup device is determined by the frame rate of images per unit time. As the frame rate is higher, the power consumption is larger. According to the present embodiment, the observation time can be reduced as compared to the conventional one, so that the frame rate in the image pickup unit can be increased by the reduction.

Therefore, image signals indicating higher-quality endoscopic images can be obtained, resulting in examination and observation with higher accuracy.

In the conventional capsule endoscope system, after the observation, the capsule endoscope 2 is naturally removed from the body by gravity and peristalsis. Accordingly, an unexpected accident may occur until the removal of the capsule endoscope. For example, the body cavity may be clogged with the capsule endoscope. When the unexpected accident happens, the capsule endoscope 2 has to be removed from the body by, for example, a surgical operation. The surgical operation is a heavy burden on the subject. According to the present embodiment, however, the attitude of the capsule endoscope 2 is controlled or the capsule endoscope 2 is forced to be moved, so that the capsule endoscope 2 can be expelled from the body with more reliability and safety.

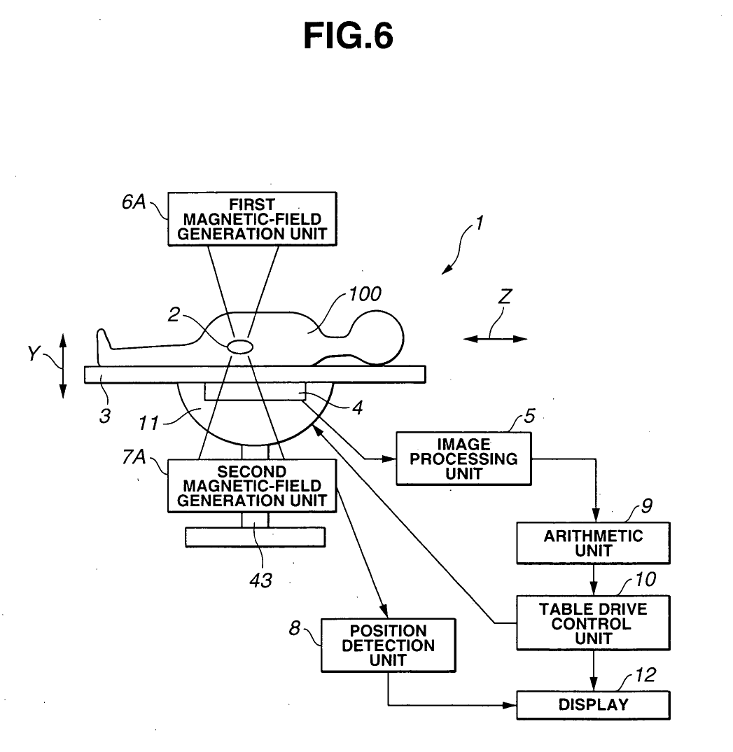

According to the present embodiment, the magnetic-field generation units 6 and 7 apply magnetic forces to the subject 100 on both the sides of the examination table 3. The application of magnetic forces is not limited to this arrangement. For example, according to a modification of the present embodiment, as shown in FIG. 6, first and second magnetic-field generation units 6A and 7A can be arranged vertically so as to apply magnetic forces to the subject 100 on the examination table 3 from above and below.

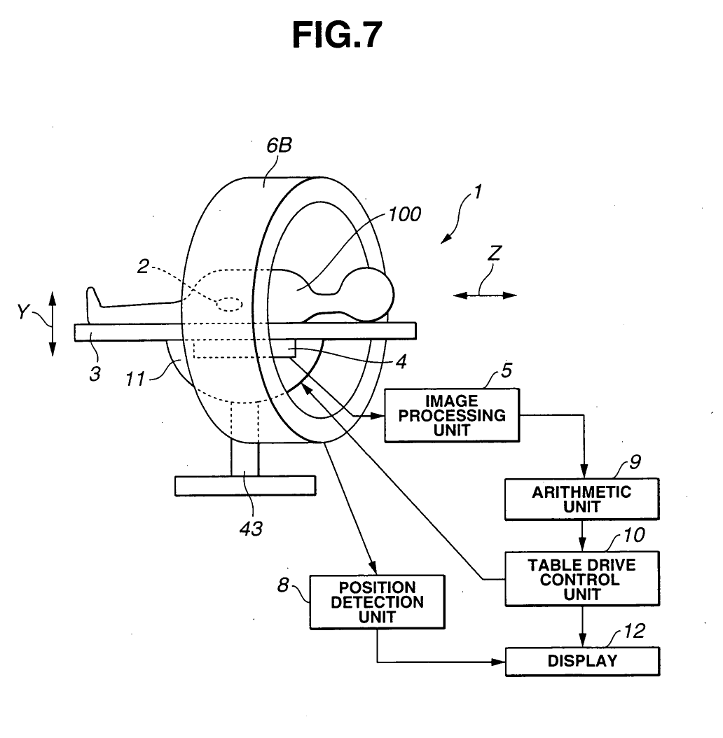

According to the present embodiment, one pair of magnetic-field generation units, namely, the first and second magnetic-field generation units 6 and 7 are arranged as magnetic-field generating means for applying a magnetic field. The structure of the magnetic-field generating means is not limited to the above. According to another modification of the present embodiment, the magnetic-field generating means can be formed so as to surround the subject 100 or the examination table 3. Referring to FIG. 7, a substantially semicylinder, cylinder, or circular magnetic-field generation ring 6B can be used. The shape of the ring is similar to those used in MRI devices and CT scanning devices. Using the ring-shaped magnetic-field generating means, a magnetic field can be generated more stably. Thus, the movement of the capsule endoscope 2 can be controlled with more reliability.

As another way of applying a magnetic field, the magnetic fields can be applied from both sides as shown in FIG. 2 and also be applied from above and below as shown in FIG. 6. In other words, the magnetic fields can be applied from four positions. In this case as well, the application of the magnetic fields with the highest intensity is focused on one point, so that the magnetic fields are generated so as to be focused on this one point.

According to the foregoing embodiment, the magnetic-field generating means includes one pair of magnetic-field generation units 6 and 7. In the above way of applying magnetic fields from four positions, two other magnetic-field generation units 6 and 7 are freely movable in the direction X and Z in FIG. 3. In this arrangement, the four magnetic-field generation units can be controlled so that the magnetic fields can always be generated in the vicinity of the capsule endoscope 2. Consequently, the amount of generated magnetic fields required for the operation of the present system can always be properly controlled, resulting in efficient operation. This arrangement also contributes to miniaturized magnetic-field generation units.

According to the foregoing embodiment, the table drive unit 11 and the table drive control unit 10 are arranged. The table drive unit 11 moves the examination table 3 in the direction Z in FIGS. 2 and 3 relative to the magnetic-field generation units 6 and 7. The table drive control unit 10 controls the table drive unit 11. The arrangement of the examination table 3, the table drive unit 11, and the table drive control unit 10 is not limited to the above. Any arrangement can be used so long as the examination table 3 is moved relative to the magnetic-field generation units 6 and 7.

For example, the examination table 3 can be fixed and the magnetic-field generation units 6 and 7 are movable in the direction Z, X, or Y. In this arrangement, instead of the table drive unit 11 for moving the examination table 3 and the table drive control unit 10 for controlling the unit 11, moving means for moving the magnetic-field generation units 6 and 7 in a predetermined direction and a drive control unit for driving the moving means are arranged. Thus, the same advantages as those of the embodiment can be obtained.

Generally, the subject 100 lies on his/her back on the examination table 3. At this time, the necessary amount of applied magnetic fields may vary depending on the physique of the subject 100. Therefore, the amount of generated magnetic fields may be changed by controlling the intensity of magnetic fields generated by the magnetic-field generation units 6 and 7. In the above arrangement, the amount of generated magnetic fields can be adjusted so as to be appropriate to each of subjects 100 having various physiques. Thus, the operation can be performed with higher efficiency and the influence of magnetic fields can be minimized.

According to the foregoing embodiment, as shown in the first and second arrangement examples in FIGS. 4 and 5, the magnetic member made of a magnetic material is formed in at least one portion of the capsule endoscope 2. Thus, the capsule endoscope 2 can be guided to a desired position by the action of the magnetic forces. In the second arrangement example in FIG. 5, the magnetic members are made of a hard magnetic material and the poles of the magnetic fields generated by the magnetic-field generation units 6 and 7 are controlled, so that the attitude of the capsule endoscope 2 can be controlled with more reliability.

Specifically speaking, the poles of magnetic fields generated by the pair of magnetic-field generation units 6 and 7 are controlled in accordance with the poles of the hard magnetic members arranged in the capsule endoscope 2. Consequently, the capsule endoscope 2 can be rotated in, for example, the direction R shown by the arrow in FIG. 5.

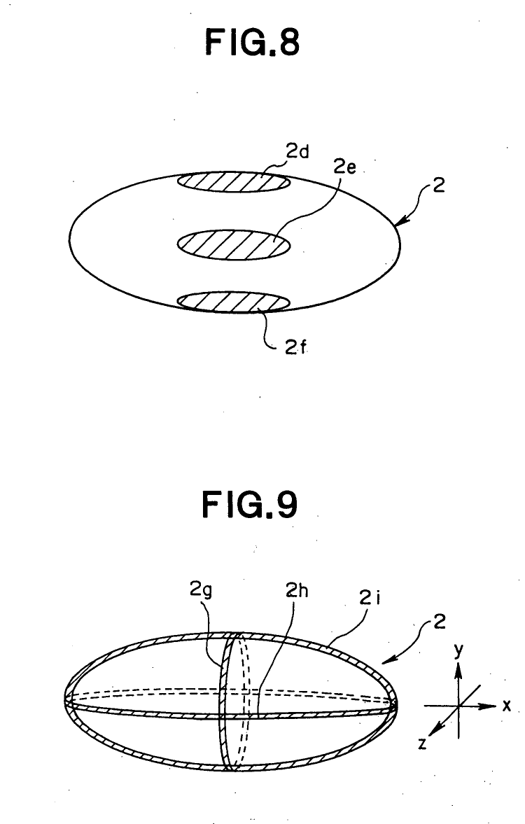

According to the foregoing embodiment, as described with reference to FIGS. 4 and 5, the magnetic member is arranged in at least one portion of the capsule endoscope 2. The arrangement of the magnetic member in the capsule endoscope 2 is not limited to the above. For example, referring to FIGS. 8 and 9, magnetic fields can be generated using the action of electromagnets.

Specifically speaking, FIG. 8 shows a first example of the arrangement of magnetic coils serving as magnetic-field generating members. The magnetic coils are arranged at predetermined positions in the capsule endoscope 2. In the first arrangement example, at least one magnetic coil may be arranged. A plurality of magnetic coils can also be arranged. Referring to FIG. 8, magnetic coils 2d, 2e, 2f, . . . are arranged at predetermined positions, namely, at regular intervals in the middle on the surface of the capsule endoscope 2.

FIG. 9 shows a second example of the arrangement of the magnetic coils serving as magnetic-field generating members. The magnetic coils are arranged at predetermined positions on the outer or inner surface of the capsule endoscope 2. According to the second arrangement example, magnetic coils 2g, 2h, and 2i are arranged so as to correspond to three planes perpendicular to the x axis, the y axis, and the z axis, respectively. The x, y, and z axes perpendicularly intersect one another.

In other words, referring to FIG. 9, the magnetic coil 2g, corresponding to the plane perpendicular to the x axis, is arranged in the middle in the longitudinal direction on the surface of the capsule endoscope 2. The magnetic coil 2h corresponds to the plane perpendicular to the y axis. The magnetic coil 2i corresponds to the plane perpendicular to the z axis. The coils 2h and 2i are also arranged in the respective predetermined positions on the surface of the capsule endoscope 2. The respective planes intersect one another.

When the capsule endoscope 2 with the above structure is used, current supply is controlled so that a current flows through at least one predetermined magnetic coil. Thus, the capsule endoscope 2 also generates a magnetic field. When the capsule endoscope 2 exists in the magnetic fields generated by the magnetic-field generation units 6 and 7, the capsule endoscope 2 is affected by the magnetic fields. Consequently, the attitude and movement of the capsule endoscope 2 can be easily controlled by remote operation.

In this case, power supply to the magnetic coils is generally conducted by the power supply unit (not shown) included in the capsule endoscope 2. An external power supply device (not shown) may be arranged separately from the above power supply unit. Power can be supplied from the external power supply device to the capsule endoscope 2 through predetermined radio communication means such as the receiving unit 4 or a power transmission unit (not shown), which is separated from the receiving unit 4. In other words, power supply can also be performed in a wireless manner.

In this case, the power supply unit (not shown) in the capsule endoscope 2 is allowed to control power supply to internal circuits in the capsule endoscope 2 and is also allowed to serve as power receiving means for receiving power from the above-mentioned external power supply device.

According to this arrangement, the magnetic coils are arranged in the capsule Endoscope 2 and a current is supplied to at least one magnetic coil in the predetermined case, so that the capsule endoscope 2 generates a magnetic field. In this case, after the capsule endoscope 2 is introduced into the body of the subject, the position of the capsule endoscope 2 can be detected from the outside.

As mentioned above, the position of the capsule endoscope 2 with the magnetic coils can be detected. In addition to the above, the position of the capsule endoscope 2 including the magnetic members made of a hard magnetic material in FIG. 5 can be similarly detected so long as the magnetic members generate magnetic fields.

In the capsule endoscope 2 with the magnetic coils, the action of electric power generation, caused by externally applying an intense magnetic field, can be used. Thus, power required for the operation of the capsule endoscope 2 can be supplied by the above action.

Pulse signals including high-frequency pulses, namely, alternating magnetic fields can be applied as magnetic fields externally applied to the capsule endoscope 2. Consequently, the movement of the capsule endoscope 2 can be controlled and the action of electric power generation can simultaneously be realized to supply power to internal electric circuits in the capsule endoscope 2. In this case, the system can be designed such that electricity is produced during the application of high-frequency pulses and the movement of the capsule endoscope 2 is controlled while the high-frequency pulses are not applied.

So long as magnetic fields are externally applied as pulse signals, when magnetic forces are applied to the capsule endoscope 2, the capsule endoscope 2 can be moved and electricity can be produced. When magnetic forces are not applied to the capsule endoscope 2, the position of the capsule endoscope 2 can be detected using the action of the magnetic coils included in the capsule endoscope 2.

Thus, the above-mentioned structure can contribute to improved accuracy of position detection. Further, the efficiency of power consumption can be increased, resulting in lower power consumption.

Further, a function of mounting a drug can be provided for the capsule endoscope of the capsule endoscope system according to the present embodiment. A function of treating or picking up cells can also be provided for the capsule endoscope. In the above structure, since the movement of the capsule endoscope 2 does not depend on peristalsis, it is expected that accurate treatment or picking of cells is conducted by controlling magnetic forces. According to the present invention, therefore, treatment and picking can be performed with more reliability and safety.

Having described the preferred embodiment of the invention referring to the accompanying drawings, it should be understood that the present invention is not limited to the precise embodiment and various changes and modifications thereof could be made by one skilled in the art without departing from the spirit or scope of the invention as defined in the appended claims.

What is claimed is:

1. A capsule endoscope system comprising:
a capsule endoscope, of which movement is controlled by a magnetic field externally applied;
at least one magnetic-field generating means for generating a magnetic field focused on one point to control the movement of the capsule endoscope travelling in a body cavity of a subject lying down on an examination table; and
moving means for moving the examination table relative to the magnetic-field generating means.

2. The capsule endoscope system according to claim 1, wherein a magnetic-field generating member is arranged in at least one portion of the capsule endoscope.

3. The capsule endoscope system according to claim 2, wherein the magnetic-field generating member includes a magnetic material.

4. The capsule endoscope system according to claim 3, wherein the magnetic material includes a hard magnetic material.

5. The capsule endoscope system according to claim 3, wherein the magnetic material includes a soft magnetic material.

6. The capsule endoscope system according to claim 2, wherein the magnetic-field generating member includes a magnetic coil arranged in the interior of the capsule endoscope.

7. The capsule endoscope system according to claim 6, wherein
a plurality of magnetic coils are arranged in the capsule endoscope, and
a current is selectively supplied to at least one of the magnetic coils in a time series manner.

8. The capsule endoscope system according to claim 1, wherein the magnetic-field generating means electrically generates a magnetic field such that the magnetic field is controllable.

9. The capsule endoscope system according to claim 8, wherein the magnetic-field generating means is controlled such that a magnetic field is intermittently applied.

10. The capsule endoscope system according to claim 9, wherein the magnetic field, generated by the magnetic-field generating means, includes an alternating magnetic field.

11. The capsule endoscope system according to claim 1, wherein the magnetic-field generating means are arranged in both sides of the subject to apply magnetic fields to the subject from both the sides.

12. The capsule endoscope system according to claim 1, wherein the magnetic-field generating means are arranged above and below of the subject to apply magnetic fields to the subject from above and below.

13. The capsule endoscope system according to claim 1, wherein the magnetic-field generating means applies a magnetic field to the subject so as to surround the subject.

14. The capsule endoscope system according to claim 1, wherein after observation of a region through the capsule endoscope, the application of the magnetic field generated by the magnetic-field generating means is interrupted.

15. The capsule endoscope system according to claim 1, wherein the moving means moves the examination table relative to the magnetic-field generating means to guide the capsule endoscope from the mouth or anus of the subject to an object region to be observed.

16. The capsule endoscope system according to claim 1, wherein the moving means moves the examination table relative to the magnetic-field generating means to remove the capsule endoscope from the mouth or anus of the subject.

17. The capsule endoscope system according to claim 1, further comprising:
a display device for displaying the position of the capsule endoscope.
